(19) United States
(12) Patent Application Publication
(10) Pub. No.: US 2022/0304814 A1
(43) Pub. Date: Sep. 29, 2022

(54) Prostheses for Stabilizing Bone Structures

(71) Applicant: Tenon Medical, Inc., Los Gatos, CA (US)

(72) Inventor: Richard S. Ginn, Gilroy, CA (US)

(21) Appl. No.: 17/834,223

(22) Filed: Jun. 7, 2022

Related U.S. Application Data
(63) Continuation-in-part of application No. 17/463,831, filed on Sep. 1, 2021.
Application No. 17/463,831 is a continuation-in-part of application No. 13/857,977, filed on Apr. 5, 2013, now Pat. No. 11,273,042.
Application No. 13/857,977 is a continuation of application No. 13/192,289, filed on Jul. 27, 2011, now abandoned.
(60) Provisional application No. 61/368,233, filed on Jul. 27, 2010.

(51) Int. Cl. A61F 2/30; A61B 17/16; A61B 17/70; A61B 17/88; A61F 2/44; A61B 5/055; A61B 5/06; A61B 6/03; A61B 6/00; A61B 8/08; A61B 34/20; A61F 2/46

(52) U.S. Cl. CPC A61F 2/30988; A61B 17/1604; A61B 17/1671; A61B 17/70; A61B 17/7055; A61B 17/8858; A61F 2/4455; A61B 17/7074; A61B 5/055; A61B 5/062; A61B 6/032; A61B 6/485; A61B 8/0841; A61B 34/20; A61B 17/1664; A61F 2/4601; A61F 2/4611; A61F 2002/30121; A61F 2002/30123; A61B 2576/00; A61F 2002/30166; A61F 2002/30593; A61F 2002/30995

(57) ABSTRACT

Prostheses are described for stabilizing dysfunctional bone structures. The prostheses have proximal and distal ends, and an expandable mid-region disposed therebetween. The expandable mid-region includes a plurality of deflectable elongate members that are configured and adapted to transition from a compressed configuration to a deflected configuration when released from a deployment apparatus, whereby the plurality of deflectable elongate members deflects outwardly when the elongated member is inserted into a pilot opening of a dysfunctional bone structure, whereby the plurality of elongate members exerts a retaining force on the internal surface of the pilot opening and secures the elongated member in the pilot opening and, thereby, the dysfunctional bone structure.

### CROSS-REFERENCES TO RELATED APPLICATIONS

This application is a continuation-in-part application of U.S. patent application Ser. No. 17/463,831, filed Sep. 1, 2021, which is a continuation-in-part application of U.S. patent application Ser. No. 13/857,977, filed Apr. 5, 2013, now U.S. Pat. No. 11,273,042, which is a continuation application of U.S. patent application Ser. No. 13/192,289, filed Jul. 27, 2011, now abandoned, which claims the benefit of U.S. provisional patent application Ser. No. 61/368,233, filed Jul. 27, 2010.

### FIELD OF THE INVENTION

The present invention relates to methods and apparatus for stabilizing bone structures, including articulating bone structures, such a sacroiliac (SI) joints and intervertebral joints.

### BACKGROUND OF THE INVENTION

As is well known in the art, there are a multitude of skeletal disorders that often necessitate stabilizing bone structures, such as skeletal member, i.e., bone, fractures and dysfunctional non-articulating and articulating bone structures, i.e., joints, such as synovial joint degeneration, and like disorders.

Various prostheses have thus been developed to stabilize bone structures. As is also well known in the art, the most common type of prostheses that have been employed to stabilize damaged or diseased and, hence, dysfunctional bone structures are bone screws and pins. The noted prostheses, which typically comprise a solid elongated structure, are often employed in combination with other fastening implements (e.g., bone plates).

More recently, considerable effort has been directed to developing improved threaded and non-threaded prostheses, i.e., screw and pin structures, and other prosthesis configurations for stabilizing bone structures; particularly, dysfunctional sacroiliac (SI) joints and intervertebral joints of the spine (e.g., adjacent vertebrae).

Referring now to FIG. 1, there is shown a schematic illustration of a human pelvic region showing the articulating bone structures, i.e., sacroiliac (SI) joints, thereof. As illustrated in FIG. 1, the SI joint 6 is defined by the interface between the articular surfaces of the sacrum 2 and the ilium 4. Thus, the SI joint 6 is defined by (and, hence, comprises) portions of the sacrum 2 and ilium 4.

Generally, the articular surfaces of the sacrum 2 and the ilium 4 that define the SI joint 6 comprise cortical bone 8, which is more compact, dense, and hard relative to softer trabecular bone 10, which, as further illustrated in FIG. 1, is disposed in the interior regions of the sacrum and ilium 2, 4.

As is well established, the SI joint performs several seminal biomechanical functions. The primary functions of the SI joint are to attenuate loads exerted on the upper body and to distribute the loads to the lower extremities. The SI joint also functions as a shock absorber for loads exerted on spine.

As is also well established, the noted loads and, hence, forces exerted on the SI joint can adversely affect the biomechanical functions of the SI joint, which can, and often will, result in SI joint dysfunction—an often-overlooked musculoskeletal pathology associated with lower back pain.

SI joint dysfunction, and pain associated therewith, can be caused by various SI joint abnormalities and/or disorders, including traumatic fracture dislocation of the pelvis, degenerative arthritis, sacroiliitis, i.e., an inflammation or degenerative condition of the sacroiliac joint; osteitis condensans ilii, and other degenerative conditions of the SI joint structures.

As is also well established, loads and, hence, forces exerted on the spine can similarly adversely affect the biomechanical functions of the spine, which can, and often will, result in intervertebral joint dysfunction.

Intervertebral joint dysfunction, and pain associated therewith, can be caused by various abnormalities and/or disorders, including herniated discs, traumatic fracture of the spine, degenerative disc disease, degenerative arthritis, and other degenerative conditions of intervertebral joint structures.

Various non-surgical methods, such as administration of pharmacological agents, e.g., the corticosteroid prednisone, have been developed and employed to treat SI and intervertebral joint dysfunction. However, such non-surgical methods have garnered limited success.

Considerable effort has thus recently been directed to developing improved surgical methods and apparatus, i.e., prostheses, to treat SI and intervertebral joint dysfunction. Such prostheses are typically configured and adapted to stabilize (i.e., reinforce or modulate articulation of) the dysfunctional bone structure, i.e., joint, via fixation or fusion of bones associated therewith.

Although several conventional surgical bone structure stabilization methods and associated bone structure prostheses have effectively ameliorated pain associated with bone structure dysfunction, there remains many disadvantages associated with the conventional methods and associated bone prostheses.

A major disadvantage associated with many conventional surgical bone structure stabilization methods is that the surgeon is typically required to make a substantial incision in and through the skin and tissues of a subject to access the dysfunctional bone structure. Often referred to as “open surgery” methods, these surgical methods have the attendant disadvantages of requiring general anesthesia and often involve increased operative time, pain, hospitalization, and recovery time due to the extensive soft tissue damage. There is also an increased probability of post-surgical complication associated with open surgery methods, such as nosocomial infection.

Minimally-invasive surgical methods to stabilize dysfunctional bone structures; particularly, SI and intervertebral joints, have thus been developed to address the noted disadvantages associated with open surgery methods. Although conventional minimally-invasive bone structure stabilization methods, such as the intervertebral bone structure (i.e., facet joint) stabilization methods disclosed in U.S. Pub. No. 2009/0076551 to Petersen, have garnered some success in relieving pain associated with bone structure, i.e., joint dysfunction, and have effectively addressed many of the disadvantages associated with open surgery methods, there similarly remains many disadvantages associated with conventional minimally-invasive bone structure stabilization methods.

A major disadvantage associated with many conventional minimally-invasive bone structure stabilization methods and associated bone structure prostheses, such as the intervertebral bone structure stabilization methods and prostheses disclosed in U.S. Pub. No. 2009/0076551 to Petersen, is that pre-existing bone structure abnormalities can lead to displacement of the implanted prostheses, which can, and often will result in damage to surrounding bone and soft tissue structures.

An additional disadvantage associated with many conventional minimally invasive bone structure stabilization methods is that the prostheses associated therewith are often prone to failure due to ineffective engagement of the prostheses to the dysfunctional bone structure, which can, and often will, result in displacement of the prostheses in the dysfunctional bone structure.

It would thus be desirable to provide improved bone structure prostheses that substantially reduce or eliminate the disadvantages associated with conventional bone structure stabilization methods and prostheses.

It is therefore an object of the invention to provide improved bone structure prostheses that substantially reduce or eliminate the disadvantages associated with conventional bone structure stabilization methods and prostheses.

It is another object of the invention to provide improved bone structure prostheses that can be readily employed to stabilize dysfunctional bone structures, including individual skeletal members and non-articulating and articulating bone structures; particularly, dysfunctional SI and intervertebral joints.

It is another object of the invention to provide improved bone structure prostheses, which, when implanted in a dysfunctional non-articulating or articulating bone structure, such as a dysfunctional SI or intervertebral joint, effectively ameliorate pain associated with bone structure dysfunction.

It is another object of the invention to provide improved bone structure prostheses that can readily be employed in minimally-invasive bone structure stabilization methods and provide secure engagement to bone structures.

It is another object of the invention to provide improved bone structure prostheses that possess optimal structural properties.

It is another object of the invention to provide improved bone structure prostheses that can be readily employed to stabilize individual bone structures, i.e., skeletal members, via fixation or fusion.

It is yet another object of the invention to provide improved bone structure prostheses that facilitate remodeling of damaged osseous tissue and regeneration of new osseous tissue and osseous tissue structures when engaged to bone structures.

### SUMMARY OF THE INVENTION

The present invention is directed to bone structure prostheses for stabilizing dysfunctional bone structures, including individual skeletal members, such as a tibia and femur, and non-articulating and articulating bone structures; particularly, dysfunctional SI and intervertebral joints.

In one embodiment of the invention, the bone structure prosthesis comprises an elongated member adapted to be inserted into a pilot opening in a dysfunctional bone structure, the pilot opening comprising an internal surface,

the elongated member comprising a proximal end and a distal end disposed opposite the proximal end, and an expandable mid-region disposed between the proximal and distal ends,

the elongated member further comprising a longitudinal axis,

the expandable mid-region comprising a plurality of deflectable elongate members configured and adapted to transition from a compressed configuration to a deflected configuration, whereby the plurality of deflectable elongate members deflects outwardly in relation to the longitudinal axis of the elongated member, and whereby, when the elongated member is inserted into the pilot opening in the dysfunctional bone structure, the plurality of elongate members exerts a retaining force on an internal surface of the pilot opening, whereby the elongated member is engaged to the pilot opening and, thereby, the dysfunctional bone structure.

In some embodiments of the invention, the transition of the plurality of deflectable elongate members from the compressed configuration to the deflected configuration is achieved by restraining the elongated member in the compressed configuration in a deployment apparatus and thereafter discharging the elongated member out of the deployment apparatus.

In some embodiments of the invention, the elongated member comprises a shape memory alloy.

In some embodiments, the shape memory alloy comprises a superelastic nickel-titanium (Ni—Ti) alloy.

In the noted embodiments, the transition of the plurality of deflectable elongate members from the compressed configuration to the deflected configuration is induced by the insertion of the elongated member into the pilot opening in the dysfunctional bone structure, whereby the elongated member is subjected to a core temperature of the subject above a crystalline structure transition temperature of the elongated member.

In some embodiments of the invention, the elongated member comprises an outer coating.

In some embodiments of the invention, the outer coating comprises a biocompatible adhesive composition.

In some embodiments, the adhesive composition comprises a poly(L-glutamic acid)-based composition, poly(γ-glutamic acid)-based composition, poly(alkyl cyano acrylate)-based composition, or polyacrylic acid-based composition.

In some embodiments, the outer coating comprises an osteogenic composition.

In some embodiments, the osteogenic composition comprises a poly(glycerol sebacate) (PGS) based composition.

In some embodiments, the osteogenic composition comprises a bone morphogenic protein (BMP).

In some embodiments, the BMP comprises BMP-1, BMP2a, BMP2b, BMP3, BMP4, BMP5, BMP6, BMP7, or BMP8a.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 is a schematic illustration of a human pelvic region from an anteroposterior (AP) perspective showing the SI joints thereof;

FIG. 2 is an illustration of a SI joint showing lateral and posterior approaches to the SI joint, in accordance with the invention;

FIG. 3A is a magnetic resonance image (Mill) of a dysfunctional SI joint from an AP perspective;

FIG. 3B is an Mill of a dysfunctional intervertebral joint from a lateral perspective;

FIG. 3C is an Mill of a fractured femur from an AP perspective;

FIG. 4A is a perspective view of one embodiment of a bone structure prosthesis, in accordance with the invention;

FIG. 4B is a top plan view of the bone structure prosthesis shown in FIG. 4A, in accordance with the invention;

FIG. 4C is a rear plan view of the bone structure prosthesis shown in FIG. 4A, in accordance with the invention;

FIG. 4D is a front plan view of the bone structure prosthesis shown in FIG. 4A, in accordance with the invention;

FIG. 4E is a rear perspective view of the bone structure prosthesis shown in FIG. 4A, in accordance with the invention;

FIG. 4F is a front perspective view of the bone structure prosthesis shown in FIG. 4A, in accordance with the invention;

FIG. 5A is an illustration of a SI joint showing one embodiment of a pilot SI joint opening, in accordance with the invention;

FIGS. 5B and 5C are illustrations of further embodiments of SI joint openings, in accordance with the invention;

FIG. 6A is a perspective view of another embodiment of a bone structure prosthesis, in accordance with the invention;

FIG. 6B is a perspective view of the bone structure prosthesis shown in FIG. 6A in an expanded configuration, in accordance with the invention;

FIG. 7A is a front plan view of one embodiment of a bone structure prosthesis deployment apparatus, in accordance with the invention; and

FIG. 7B is a partial front plan view of the alignment member of the bone structure prosthesis deployment apparatus shown in FIG. 7A, in accordance with the invention.

### DETAILED DESCRIPTION OF THE PREFERRED EMBODIMENT

Before describing the present invention in detail, it is to be understood that this invention is not limited to particularly exemplified apparatus, systems, structures or methods as such may, of course, vary. Thus, although a number of apparatus, systems, structures and methods similar or equivalent to those described herein can be used in the practice of the present invention, the preferred apparatus, systems, structures and methods are described herein.

It is also to be understood that, although the present invention is primarily described and illustrated in connection with bone structure prostheses and methods for stabilizing dysfunctional sacroiliac (SI) joints, the invention is not limited to such prostheses and methods. According to the invention, the apparatus, systems, structures and methods of the invention can also be employed to stabilize other articulating bone structures, and non-articulating bone structures, such as intervertebral joints. The apparatus, systems, structures and methods of the invention can also be employed to stabilize individual skeletal members, i.e., bones, including, without limitation, spinal vertebrae, intertarsal bones, femur and the like.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

As used in this specification and the appended claims, the singular forms “a”, “an” and “the” include plural referents unless the content clearly dictates otherwise. Thus, for example, reference to “an incision” includes two or more incisions and the like.

Further, ranges can be expressed herein as from “about” or “approximately” one particular value, and/or to “about” or “approximately” another particular value. When such a range is expressed, another embodiment includes from the one particular value and/or to the other particular value. Similarly, when values are expressed as approximations, by use of the antecedent “about” or “approximately”, it will be understood that the particular value forms another embodiment. It will be further understood that the endpoints of each of the ranges are significant both in relation to the other endpoint, and independently of the other endpoint.

It is also understood that there are a number of values disclosed herein, and that each value is also herein disclosed as “about” or “approximately” that particular value in addition to the value itself. For example, if the value “10” is disclosed, then “approximately 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed then “less than or equal to 10” as well as “greater than or equal to 10” is also disclosed.

### Definitions

The terms “bone” and “bone structure” are used interchangeably herein, and mean and include any skeletal member or structure that comprises osseous tissue. The terms “bone” and “bone structure” thus mean and include complete and partial skeletal members or bone structures, including articulating and non-articulating bone structures, (e.g., vertebrae, sacrum, ilium, femur, etc.) and portions thereof.

The terms “sacroiliac joint”, “SI joint”, “sacroiliac junction” and “SI junction” are used interchangeably herein, and mean and include any region proximate to articulating regions of the sacrum and ilium bone structures and, hence, a junction between and defined by sacrum and ilium bone structures.

The terms “joint” and “junction” are used interchangeably herein, and mean and include any region proximate to non-articulating and articulating regions of bone structures and, hence, a junction between and defined by the bone structures. The terms “joint” and “junction” thus mean and include, without limitation, SI joints, intervertebral joints, facet joints, intertarsal joints including, subtalar joints, talocalcaneonavicular joints, calcaneocuboid joints; and like joint structures.

The term “dysfunctional” as used in connection with a bone structure, means and includes a physiological abnormality, disorder or impairment of a bone structure, including, but limited to, traumatic fracture and/or dislocation of a bone structure, e.g., SI joint, vertebrae, sacrum, ilium, femur, etc., degenerative arthritis, and/or an inflammation or degenerative condition of a bone structure.

The terms “articular surface” and “articulating surface” are used interchangeably herein in connection with bone structures, and mean and include a surface of a bone structure that forms an articulating junction with an adjacent bone structure, e.g., the articular surfaces of the sacrum and ilium bone structures.

The terms “fusion”, “arthrodesis”, and “fixation” are used interchangeably herein in connection with bone structures, and mean and include partial or complete immobilization of bone structures.

The term “stabilization”, as used herein, means and includes reinforcing, e.g., supporting, or modulating motion of bone structures. The term “stabilization”, thus, in some instances, means and includes fusion, arthrodesis, and fixation of adjacent bone structures, such as articular bone structures, and portions of a fractured bone structure, e.g., fractured femur.

The term “prosthesis”, as used herein in connection with bone structures, means and includes a system or apparatus configured and adapted to stabilize or modulate motion of bone structures.

The term “biodegradable”, as used herein, means the ability of a material; particularly, a polymer or adhesive, to breakdown and be absorbed within the physiological environment of a joint and/or a structure associated therewith, including sacrum and ilium bone structures, by one or more physical, chemical, or cellular processes.

Biodegradable polymers, according to the invention, thus include, without limitation, polylactide polymers (PLA), copolymers of lactic and glycolic acids, including poly(lactic-co-glycolic) acid (PLGA) and poly(ε-caprolactone-co-L-lactic) acid (PCL-LA); glycine/PLA co-polymers, polyethylene oxide (PEO)/PLA block copolymers, acetylated polyvinyl alcohol (PVA)/polycaprolactone copolymers, poly(glycerol sebacate) (PGS) and its derivatives, including poly(glycerol-co-sebacate acrylate) (PGSA); poly(polyol sebacate) (PPS), poly(xylitol sebacate) (PXS), poly(xylitol glutamate sebacate) (PXGS), hydroxybutyrate-hydroxyvalerate copolymers, polyesters such as, but not limited to, aspartic acid and different aliphatic diols; poly(alkylene tartrates) and their copolymers with polyurethanes, polyglutamates with various ester contents and with chemically or enzymatically degradable bonds, other biodegradable nonpeptidic polyamides, amino acid polymers, polyanhydride drug carriers such as, but not limited to, poly(sebacic acid) (PSA); aliphatic-aromatic homopolymers, and poly(anhydride-co-imides), poly(phosphoesters) by matrix or pendant delivery systems, poly(phosphazenes), poly(iminocarbonate), crosslinked poly(ortho ester), hydroxylated polyester-urethanes, or the like.

Biodegradable adhesives, according to the invention, thus include, without limitation, poly(glycerol-co-sebacate acrylate) (PGSA), poly(L-glutamic acid)-based compositions, poly(γ-glutamic acid)-based compositions, poly(alkyl cyano acrylate)-based compositions, polyacrylic acid-based compositions, including polyacrylic acid crosslinked with pentaerythritol and/or allyl sucrose, polyacrylic acid crosslinked with divinyl glycol, and combinations thereof; fibrin-based compositions, collagen-based compositions, including collagen/poly(L-glutamic acid) compositions; albumin-based compositions, including BioGlue® (comprises purified bovine serum albumin (BSA) and glutaraldehyde); cyanoacrylate compositions, including butyl-2-cyanoacrylate adhesives (e.g., Indermil®, Histoacryl®, Histoacryl® Blue, and LiquiBand®) and octyl-2-cyanoacrylate adhesives (e.g., Dermabond®, SurgiSeal™, LiquiBand® Flex, and OctylSeal); poly(ethylene glycol) (PEG) based compositions, including FocalSeal®, Progel™ Duraseal™, DuraSeal™ Xact, Coseal®, and ReSure Sealant; polysaccharide-based compositions, polypeptide-based compositions, and combinations thereof.

The term “osteogenic composition”, as used herein, means and includes an agent or composition that induces or modulates an osteogenic physiological or biological process, or cellular activity, e.g., induces proliferation, and/or growth and/or remodeling and/or regeneration of bone or osseous tissue. The term “osteogenic composition”, thus, in some instances means and includes an agent or composition that promotes, induces or modulates fusion, arthrodesis, and/or fixation of bone structures.

The term “osteogenic composition” thus also means and includes, without limitation, the following osteogenic materials and compositions comprising same: demineralized bone matrix, autograft bone material, allograft bone material, xenograft bone material, polymethyl-methacrylate, calcium-based bone void filler material, including hydroxyapatite (HA) and tricalcium phosphate; and combinations or mixtures thereof.

The term “osteogenic composition” also means and includes, without limitation, the following polymer materials and compositions comprising same: poly(glycerol sebacate) (PGS), poly(glycerol-co-sebacate) acrylate (PGSA) and co-polymers, such as poly(glycerol sebacate)-co-poly(ethylene glycol) (PGS-PEG); and/or composites thereof, e.g., PGS-hydroxyapatite (HA) composites and PGS-poly(ε-caprolactone) (PGS-PCL) composites.

The term “osteogenic composition” also means and includes, without limitation, acellular extracellular matrix (ECM) derived from mammalian tissue sources.

The term “osteogenic composition” thus means and includes, without limitation, acellular ECM derived from bone or osseous tissue, small intestine submucosa (SIS), epithelium of mesodermal origin, i.e., mesothelial tissue, placental tissue, omentum tissue, and combinations thereof.

The terms “biologically active agent” and “biologically active composition” are used interchangeably herein, and mean and include agent or composition that induces or modulates a physiological or biological process, or cellular activity, e.g., promotes and/or induces proliferation, and/or growth and/or regeneration of tissue, including osseous tissue.

The terms “biologically active agent” and “biologically active composition”, as used herein, thus include agents and compositions that can be varied in kind or amount to provide a therapeutic level effective to mediate the formation or healing of osseous tissue, cartilage, and connective tissue, e.g., tendons and ligaments. The term “biologically active composition”, in some instances, thus means and includes an “osteogenic composition.”

The terms “biologically active agent” and “biologically active composition” thus mean and include, without limitation, the following bone morphogenic proteins (BMPs) and compositions comprising same: BMP-1, BMP2a, BMP2b, BMP3, BMP4, BMP5, BMP6, BMP7 (also referred to as osteogenic protein 1 (OP-1)), and BMP8a.

The terms “biologically active agent” and “biologically active composition” also mean and include, without limitation, the following biological agents and compositions comprising same: platelet derived growth factor (PDGF), an insulin-like growth factor (IGF), including IGF-1 and IGF-2; basic fibroblast growth factor (bFGF) (also referred to as FGF2), transforming growth factor-β (TGF-β), including, TGF-β01 and TGF-β2; a growth hormone (GH), parathyroid hormone (PTH, including PTH1-34), transforming growth factor-α (TGF-α), granulocyte/macrophage colony stimulating factor (GM-CSF), epidermal growth factor (EGF), growth and differentiation factor-5 (GDF-5), vascular endothelial growth factor (VEGF), angiogenin, angiopoietin-1, del-1, follistatin, granulocyte colony-stimulating factor (G-CSF), hepatocyte growth factor/scatter factor (HGF/SF), interleukin-8 (IL-8), interleukin-10 (IL-10), leptin, midkine, placental growth factor, platelet-derived endothelial cell growth factor (PD-ECGF), platelet-derived growth factor-BB (PDGF-BB), pleiotrophin (PTN), progranulin, proliferin, a matrix metalloproteinase (MMP), angiopoietin 1 (ang1), angiopoietin 2 (ang2), and delta-like ligand 4 (DLL4).

The terms “biologically active agent” and “biologically active composition” also mean and include, without limitation, the following cells and compositions comprising same: bone marrow-derived progenitor cells, bone marrow stromal cells (BMSCs), osteoprogenitor cells, osteoblasts, osteocytes, osteoclasts, committed or partially committed cells from the osteogenic or chondrogenic lineage, hematopoietic stem cells, chondrocytes, chondrogenic progenitor cells (CPCs), mesenchymal stem cells (MSCs), and embryonic stem cells.

The terms “biologically active agent” and “biologically active composition” also mean and include an “extracellular vesicle (EV)”, “exosome”, “microsome” or “micro-vesicle”, which are used interchangeably herein, and mean and include a biological structure formed from a hydrocarbon monolayer or bilayer configured to contain or encase a composition of matter.

The terms “extracellular vesicle (EV)”, “exosome”, “microsome” and “micro-vesicle” thus include, without limitation, a biological structure formed from a lipid layer configured to contain or encase biologically active agents and/or combinations thereof.

The terms “extracellular vesicle (EV)”, “exosome”, “microsome” and “micro-vesicle” also include, without limitation, EVs derived from the aforementioned cells and compositions comprising same, e.g., BMSC-derived EVs.

The terms “pharmacological agent” and “active agent” are used interchangeably herein, and mean and include an agent, drug, compound, composition, or mixture thereof, including its formulation, which provides some therapeutic, often beneficial, effect. This includes any physiologically or pharmacologically active substance (or composition comprising same) that produces a localized or systemic effect or effects in animals, including warm blooded mammals.

The terms “pharmacological agent” and “active agent” thus mean and include, without limitation, the following osteoinductive agents and compositions comprising same: icaritin, tumor necrosis factor alpha (TNF-α) inhibitors, including etanercept and infliximab, disease-modifying anti-rheumatic drugs (DMARDs), including methotrexate and hydroxychloroquine, antibiotics, anti-viral agents, steroidal anti-inflammatories, non-steroidal anti-inflammatories, anti-thrombotic agents, including anti-coagulants and anti-platelet agents; and vasodilating agents.

The terms “pharmacological agent” and “active agent” further mean and include, without limitation, the following bisphosphonate agents and compositions comprising same: risedronate (Actonel®), alendronate (Fosamax®), ibandronate (Boniva®), zoledronic acid (Reclast®), pamidronate (Aredia®), and etidronate (Didronel®).

The terms “pharmacological agent” and “active agent” further mean and include, without limitation, the following antibiotics and compositions comprising same: penicillin, carboxypenicillins, such as ticarcillin; tetracyclines, such as minocycline; gentamicin, vancomycin, ciprofloxacin, amikacin, aminoglycosides, cephalosporins, clindamycin, erythromycin, fluoroquinolones, macrolides, azolides, metronidazole, trimethoprim-sulfamethoxazole, polymyxin B, oxytetracycline, tobramycin, cefazolin, and rifampin.

The terms “anti-inflammatory” and “anti-inflammatory agent” are also used interchangeably herein, and mean and include a “pharmacological agent”, which, when a therapeutically effective amount is administered to a subject, prevents, or treats bodily tissue inflammation, i.e., the protective tissue response to injury or destruction of tissues, which serves to destroy, dilute, or wall off both the injurious agent and the injured tissues.

Anti-inflammatory agents thus include, without limitation, dexamethasone, betamethasone, prednisone, prednisolone, methylprednisolone sodium succinate, methylprednisolone, cortisone, ketorolac, diclofenac, and ibuprofen.

The terms “pharmacological agent” and “active agent” further mean and include, without limitation, the following metal-based antimicrobials and compositions comprising same: silver particles, copper particles, cobalt particles, nickel particles, zinc particles, zirconium particles, molybdenum particles, lead particles, and mixtures thereof.

As indicated above, the term “pharmacological composition”, as used herein, means and includes a composition comprising a “pharmacological agent” and “active agent”.

The term “therapeutically effective”, as used herein, means that the amount of the “pharmacological agent” and/or “pharmacological composition” and/or “biologically active agent” and/or “biologically active composition” administered is of sufficient quantity to ameliorate one or more causes, symptoms, or sequelae of a disease or disorder. Such amelioration only requires a reduction or alteration, not necessarily elimination, of the cause, symptom, or sequelae of a disease or disorder.

The terms “patient” and “subject” are used interchangeably herein, and mean and include warm blooded mammals, humans and primates; avians; domestic household or farm animals, such as cats, dogs, sheep, goats, cattle, horses and pigs; laboratory animals, such as mice, rats and guinea pigs; fish; reptiles; zoo and wild animals; and the like.

The terms “one embodiment”, “one aspect”, and “an embodiment” and “an aspect”, as used herein, means that a particular feature, structure, or characteristic described in connection with the embodiment may be included in at least one embodiment and not that any particular embodiment is required to have a particular feature, structure or characteristic described herein unless set forth in the claim.

The phrase “in one embodiment” or similar phrases employed herein do not limit the inclusion of a particular element of the invention to a single embodiment. The element may thus be included in other, or all embodiments discussed herein.

The term “substantially”, as used herein, means and includes the complete or nearly complete extent or degree of an action, characteristic, property, state, structure, item, or result to function as indicated. For example, an object that is “substantially” enclosed would mean that the object is either completely enclosed or nearly completely enclosed. The exact allowable degree of deviation from absolute completeness may in some cases depend on the specific context, such that enclosing nearly all the length of a lumen would be substantially enclosed, even if the distal end of the structure enclosing the lumen had a slit or channel formed along a portion thereof.

Use of the term “substantially” is equally applicable when used in a negative connotation to refer to the complete or near complete lack of an action, characteristic, property, state, structure, item, or result. For example, structure which is “substantially free of” a bottom would either completely lack a bottom or so nearly completely lack a bottom that the effect would be effectively the same as if it completely lacked a bottom.

The term “comprise” and variations of the term, such as “comprising” and “comprises,” means “including, but not limited to” and is not intended to exclude, for example, other components, elements or steps.

The following disclosure is provided to further explain in an enabling fashion the best modes of performing one or more embodiments of the present invention. The disclosure is further offered to enhance the understanding and appreciation for the inventive principles and advantages thereof, rather than to limit in any manner the invention. The invention is defined solely by the appended claims, including any amendments made during the pendency of this application, and all equivalents of those claims as issued.

As indicated above, the present invention is directed to bone structure prostheses for stabilizing dysfunctional bone structures.

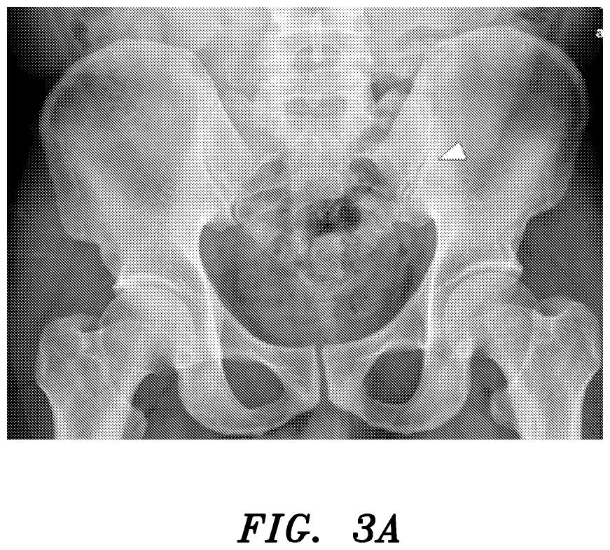

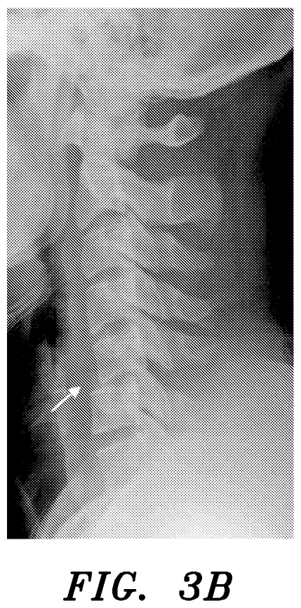

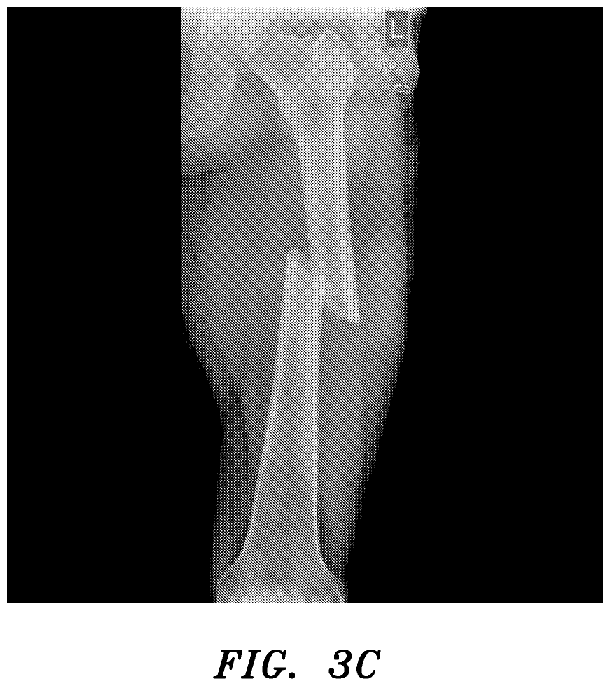

As indicated above, although the present invention is primarily described and illustrated in connection with bone structure prostheses for stabilizing dysfunctional sacroiliac (SI) joints, such as the dysfunctional SI joint shown in FIG. 3A, the invention is not limited to such prostheses and methods. Indeed, according to the invention, the bone structure prostheses of the invention can also be readily employed to stabilize other dysfunctional bone structures, including other dysfunctional articulating bone structures, such as the dysfunctional intervertebral joint shown in FIG. 3B, dysfunctional non-articulating bone structures, such as a dysfunctional pelvic girdle, and dysfunctional individual skeletal members, such as the fractured femur shown in FIG. 3C.

According to the invention, the bone structure prostheses of the invention can comprise various configurations, including pontoon shaped members and expandable members.

As indicated above, articulating bone structure, i.e., joint, stabilization methods typically comprise surgical placement of a prosthesis proximate to or in a dysfunctional bone structure via anterior, lateral, and posterior approaches to the joint.

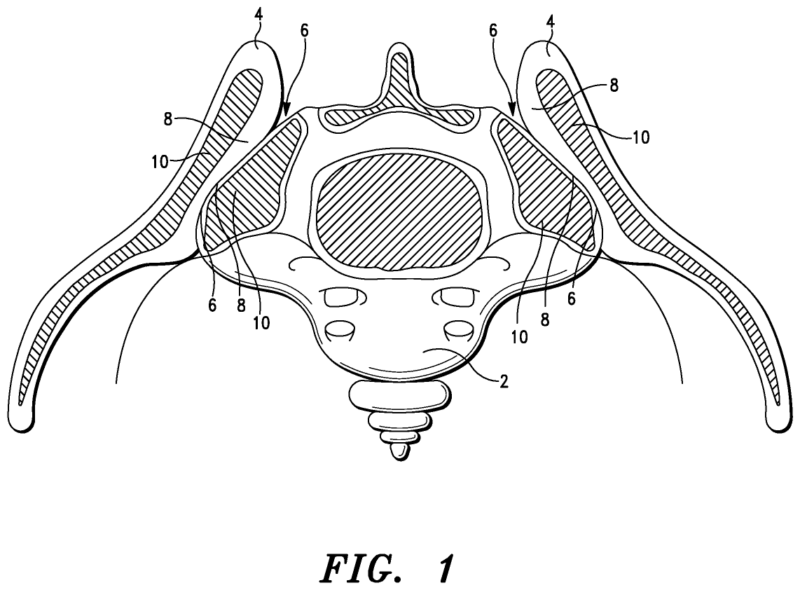

Referring back to FIG. 1, an anterior approach to an articulating bone structure, which in this instance is a SI joint 6 (and, hence, a dysfunctional SI joint), would be substantially perpendicular to the page upon which FIG. 1 is printed.

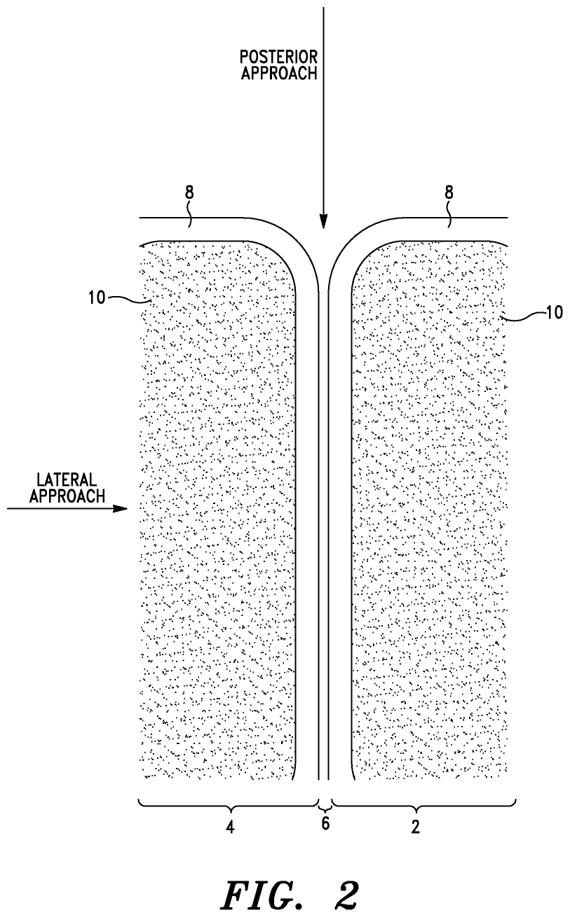

Referring now to FIG. 2 there is shown a close-up illustration of a portion of the leftmost SI joint 6 illustrated in FIG. 1, showing approximate approach vectors for lateral and posterior approaches to the SI joint 6.

In some embodiments, such as stabilizing a dysfunctional SI joint, the bone structure prostheses of the invention are configured and adapted to be implanted in dysfunctional articulating bone structures via a posterior approach.

As set forth in Co-pending U.S. application Ser. No. 17/463,831, which is expressly incorporated by reference herein, the bone structure prostheses of the invention are configured and adapted to be implanted in pilot openings in the dysfunctional articulating bone structures to stabilize the dysfunctional structures.

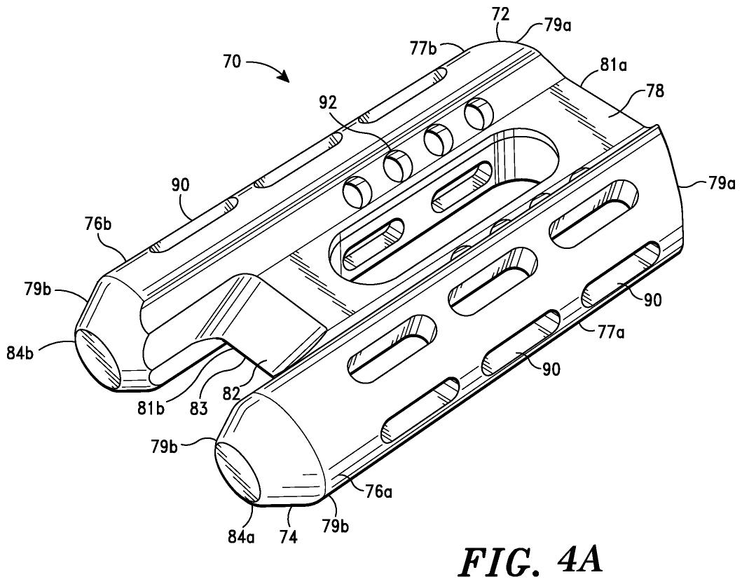

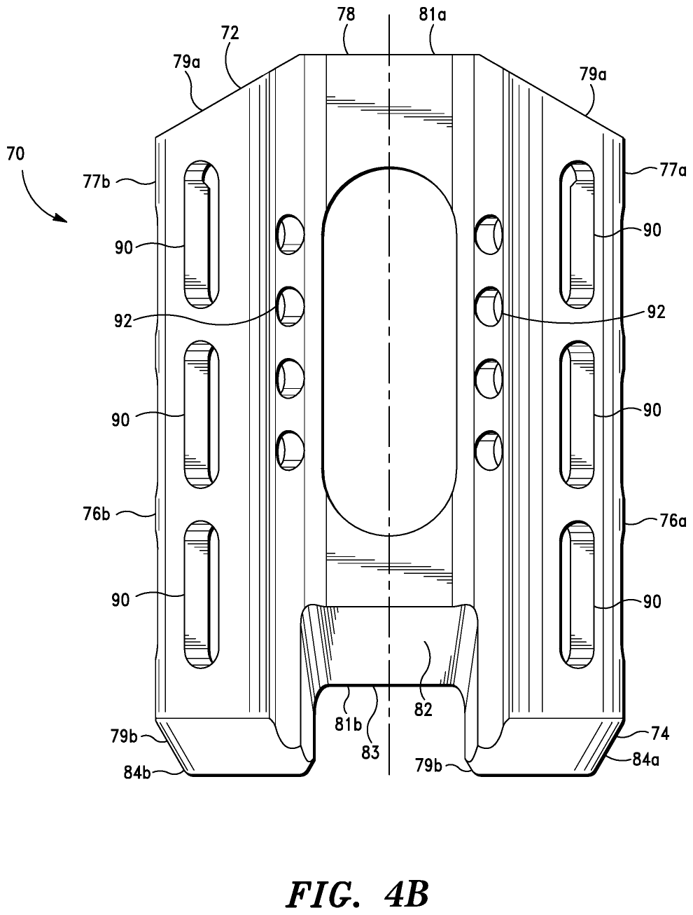

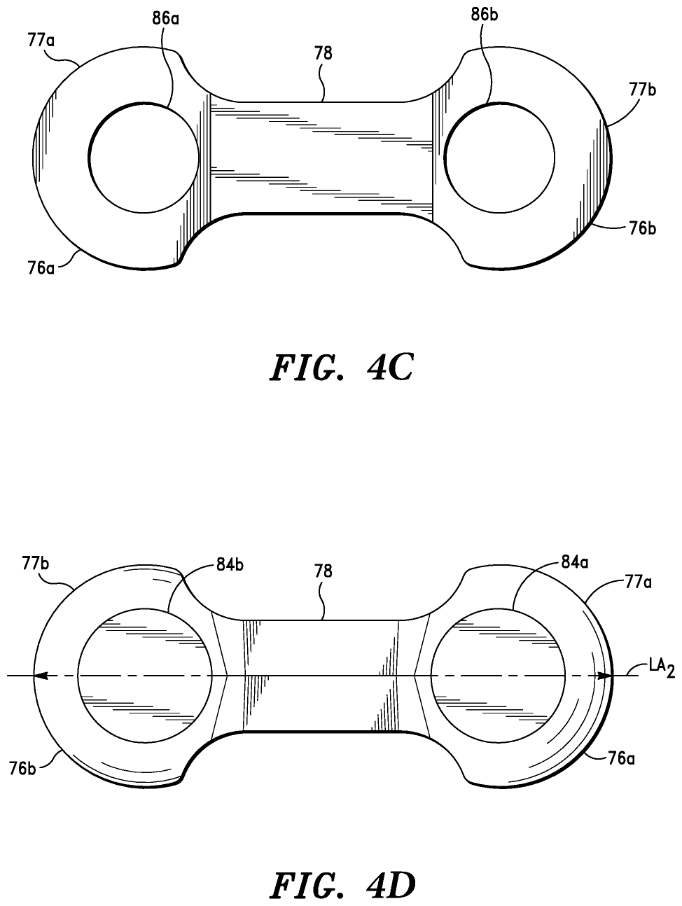

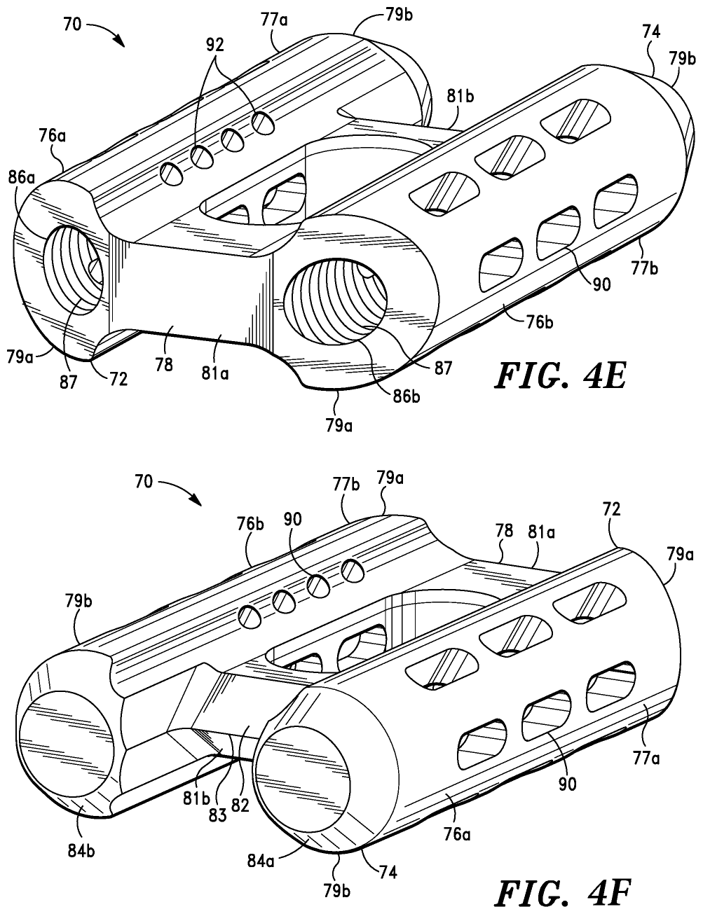

Referring now to FIGS. 4A-4H, one embodiment of a bone structure prosthesis that is specifically designed and configured to stabilize a dysfunctional SI joint will be described in detail. Although the prosthesis (denoted “70”) is described in connection with stabilizing a dysfunctional SI joint, according to the invention, the prosthesis can also be employed to stabilize other articulating and non-articulating bone structures, including individual skeletal members.

As set forth in Co-pending U.S. application Ser. No. 17/463,831, prosthesis 70 is particularly adapted, configured, and suitable for stabilizing dysfunctional SI joints via a posterior approach.

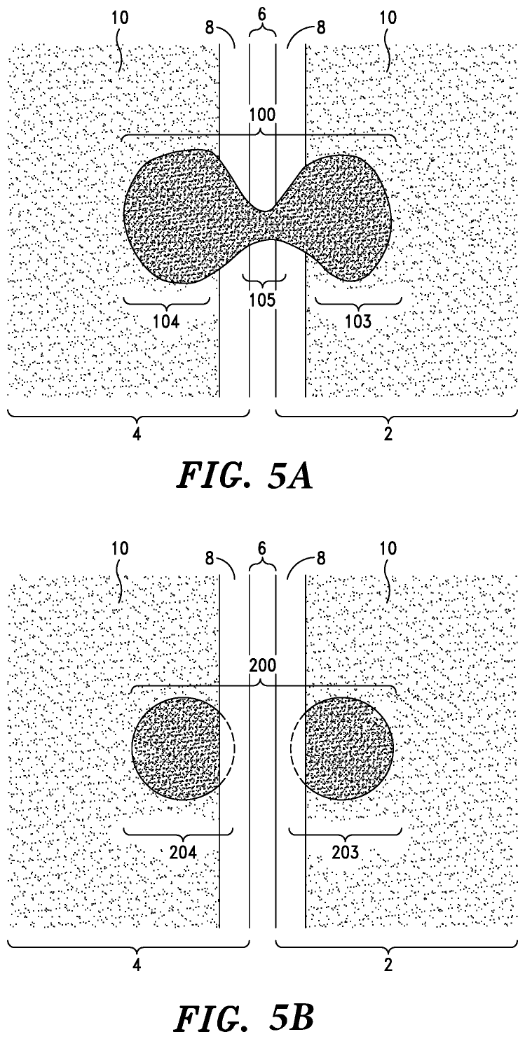

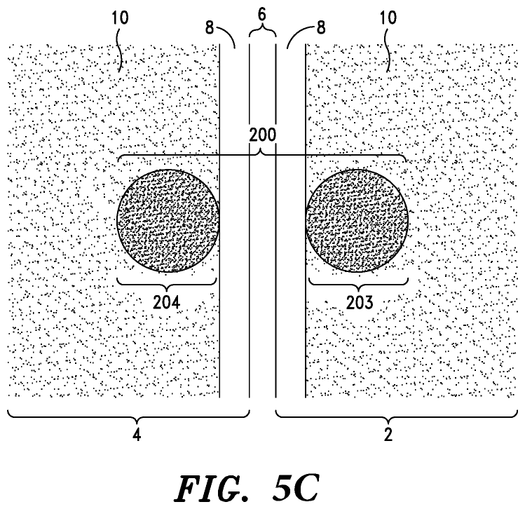

As also set forth in Co-pending U.S. application Ser. No. 17/463,831 and discussed in detail below, the prosthesis 70 is configured and adapted to be inserted into pilot openings in dysfunctional bone structures; particularly, dysfunctional SI joints, such as pilot SI joint openings 100, 200 shown in FIGS. 5A-5B and described below, and into and through articular cartilage and cortical bone (and trabecular bone), which define the joint.

As illustrated in FIGS. 4A, 4E, and 4F, the prosthesis 70 comprises a biocompatible and, hence, implantable member comprising proximal and distal ends 72, 74, and first and second elongated partially cylindrical sections 76a, 76b connected to a bridge section 78, whereby the prosthesis 70 comprises a continuous exterior surface comprising first and second partially cylindrical surface regions 77a, 77b.

As further illustrated in FIGS. 4A, 4E, and 4F, the first and second partially cylindrical sections 76a, 76b comprise proximal and distal ends 79a, 79b. The bridge section 78 similarly comprises proximal and distal ends 81a, 81b.

As set forth in Co-pending U.S. application Ser. No. 17/463,831, the prosthesis 70 can comprise any suitable length from the proximal ends 79a to the distal ends 79b of the partially cylindrical sections 76a, 76b. In some embodiments, the prosthesis 70 comprises a length in the range of 20-50 mm, more preferably, a length in the range of 30-40 mm.

As illustrated in FIGS. 4C, 4E and 4F, and FIGS. 5A and 5B, the first partially cylindrical surface region 77a preferably comprises a partially cylindrical surface region shape that corresponds to at least a portion of the first lobe region 103 of the pilot SI joint opening 100 shown in FIG. 5A and/or the sacrum guide portion 203 of the pilot SI joint opening 200 shown in FIG. 5B, and/or the second lobe region 104 of the pilot SI joint opening 100 and/or the ilium guide portion 204 of the pilot SI joint opening 200, depending on the entry position of the prosthesis 70 into the pilot SI joint openings 100, 200.

The second partially cylindrical surface region 77b similarly preferably comprises a partially cylindrical surface region shape that corresponds to at least a portion of the first lobe region 103 of the pilot SI joint opening 100 shown in FIG. 5B and/or the sacrum guide portion 203 of the pilot SI joint opening 200 shown in FIG. 5B, or the second lobe region 104 of the pilot SI joint opening 100 and/or the ilium guide portion 204 of the pilot SI joint opening 200, again depending on the entry position of the prosthesis 70 into the pilot SI joint openings 100, 200.

As illustrated in FIGS. 4A, 4B, and 4F, the distal end 81b of the bridge section 78 preferably comprises a taper region 82, which is configured and adapted to disrupt, i.e., cut into and through, articular cartilage and cortical bone 8 (and, in some aspects, trabecular bone 10), which define a SI joint.

In some embodiments, the taper region 82 comprises two angled regions that intersect at a central point 83, i.e., pointed proximate the mid-region of the bridge section 78, such as shown in FIGS. 4A and 4F.

As further illustrated in FIG. 4A, the distal ends 79b of the first and second elongated partially cylindrical sections 76a, 76b also preferably comprise tapered regions 84a, 84b, which facilitate (i) insertion of the distal ends 79b of the first and second elongated partially cylindrical sections 76a, 76b into pilot openings, such as pilot SI joint openings 100, 200 shown in FIGS. 5A and 5B.

As illustrated in FIGS. 4C and 4E, the first elongated partially cylindrical section 76a of the prosthesis 70 comprises an internal prosthesis engagement member lumen 86a that extends from the proximal end 79a of the first elongated partially cylindrical section 76a.

As illustrated in FIGS. 4C and 4E, the second elongated partially cylindrical section 76b of the prosthesis 70 also comprises an internal prosthesis engagement member lumen 86b that extends from the proximal end 79a of the first elongated partially cylindrical section 76b.

As set forth in Co-pending U.S. application Ser. No. 17/463,831, in a preferred embodiment, the internal prosthesis engagement member lumens 86a, 86b of the prosthesis 70 are sized and configured to receive a prosthesis deployment assembly that is designed and configured to engage and position the prosthesis 70 in a pilot opening and, thereby, in a dysfunctional SI joint.

Details of the preferred prosthesis deployment assembly, the engagement thereof to prosthesis 70 and positioning of prosthesis 70 in a pilot opening and, thereby, in a dysfunctional SI joint are set forth in Co-pending U.S. application Ser. No. 17/463,831.

In a preferred embodiment, the internal prosthesis engagement lumens 86a, 86b are also configured to receive agents and compositions that further facilitate adhesion of the prosthesis 70 to pilot openings of the invention; particularly, pilot SI openings 100, 200, and, thereby, bone structures. Such agents and compositions are set forth in in Co-pending U.S. application Ser. No. 17/463,831.

In a preferred embodiment, the internal prosthesis engagement lumens 86a, 86b are also configured to receive the aforementioned biologically active agents and compositions, including osteogenic agents and compositions, and pharmacological agents and compositions that promote or induce proliferation, and/or growth and/or remodeling and/or regeneration of osseous tissue and/or facilitate osseous tissue ingrowth into the prosthesis 70 when the prosthesis 70 is disposed in a pilot opening and, hence, engaged to bone structures.

Referring back to FIGS. 4A and 4B, in a preferred embodiment, the prosthesis 70 further comprises a plurality of slots 90 and holes 92, which preferably are in communication with the internal prosthesis engagement member lumens 86a, 86b.

In a preferred embodiment, the agents and compositions referenced above are adapted to extrude through the slots 90 and holes 92 of the prosthesis 70 when the prosthesis 70 is inserted in a pilot opening, such as pilot SI joint openings 100 or 200 shown in FIGS. 5A-5C, to, as indicated above, (i) further promote adhesion of the prosthesis 70 to the pilot openings and, thereby, bone structures (e.g., sacrum and/or ilium), and (ii) promote osseous or bone tissue ingrowth into the prosthesis 70 and healing of the bone structures.

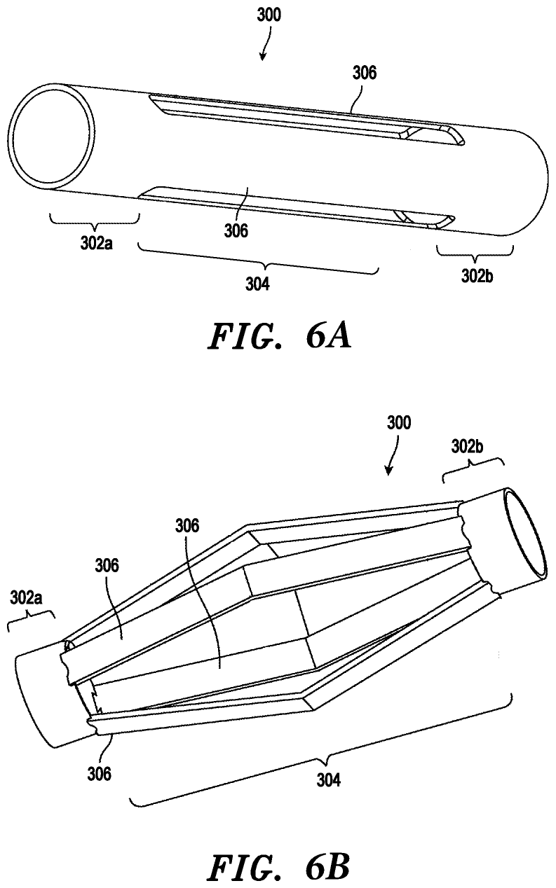

Referring now to FIGS. 6A and 6B there is shown one embodiment of an expandable bone structure prosthesis of the invention.

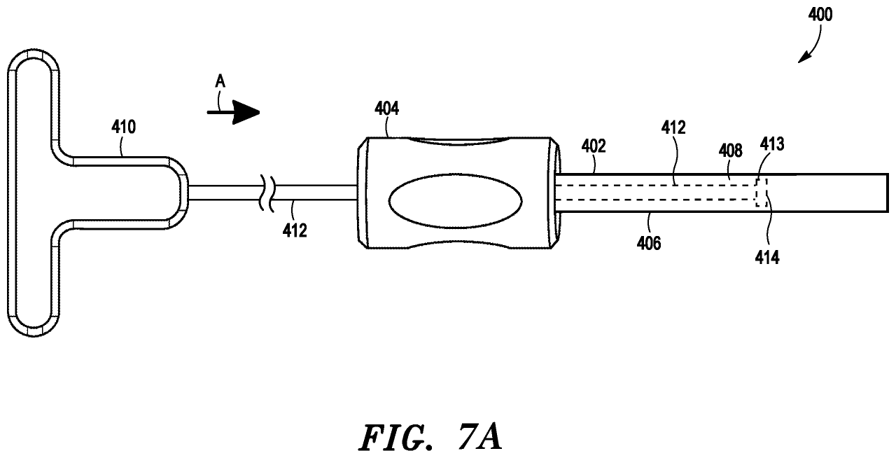

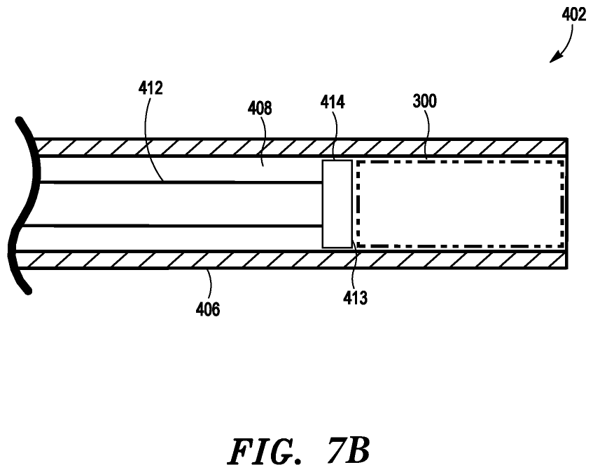

As discussed in detail below, in some embodiments of the invention, the bone structure prosthesis (denoted “300”) is similarly configured to be advanced into bone structures via a deployment apparatus, such as the deployment apparatus 400 shown in FIGS. 7A and 7B.

According to the invention, the bone structure prosthesis 300 can be deployed from a posterior or lateral approach to stabilize dysfunctional bone structures.

As set forth in priority U.S. application Ser. No. 13/857,977 and illustrated in FIGS. 6A and 6B, the expandable bone structure prosthesis 300 comprises proximal and distal end regions 302a, 302b, and an expandable mid-region 304, comprising a plurality of deflectable elongate members 306.

In a preferred embodiment of the invention, the deflectable elongate members 306 are configured and adapted to transition from a compressed configuration to an expanded configuration, wherein the elongate members 306 are deflected outwardly, as shown in FIG. 6B, when released from within a deployment apparatus, e.g., deployment apparatus 400, and/or when the proximal and distal end regions 302a, 302b of the bone structure prosthesis 300 are compressed toward each other.

In some embodiments of the invention, the deflectable elongate members 306 are positioned substantially parallel to the longitudinal axis of the bone structure prosthesis 300, as shown in FIGS. 6A and 6B. According to the invention, the deflectable elongate members 306 can also be oriented in and, hence, comprise a spiral configuration.

Referring now to FIG. 7A, there is shown one embodiment of a deployment apparatus 400 that is particularly suitable for delivering the expandable bone structure prosthesis 300 to and into a pilot opening in bone structures.

As illustrated in FIG. 7A, in a preferred embodiment, the deployment apparatus 400 comprises a two-piece structure comprising an alignment apparatus 402 and a delivery handle 410.

As illustrated in FIGS. 7A and 7B, the alignment apparatus 402 comprises an alignment handle 404 and an elongated tubular member 406 having an internal lumen 408 that extends through the elongated member 406 and alignment handle 404; the internal lumen 408 configured and adapted to receive the rod member 412 of the delivery handle 410, discussed below.

As further illustrated in FIGS. 7A and 7B, the delivery handle 410 comprises an elongated rod member 412 that is sized and configured to insert into and translate in and through the internal lumen 408 of the alignment member 402. In a preferred embodiment, the elongated rod member 412 comprises a prosthesis abutment region or end 414 disposed on the distal end 413 of the rod member 412.

In at least one embodiment of the invention, after a pilot opening is created in a bone structure, the expandable bone structure prosthesis 300 is delivered to and into the pilot opening as follows:

(i) the delivery handle 410 of the deployment apparatus 400 is retracted, whereby the rod member 412 is retracted in the internal lumen 408 of the alignment member 402;

(ii) after the delivery handle 410 is retracted, the bone structure prosthesis 300 is loaded into the deployment apparatus 400, i.e., the prosthesis 300 is inserted into the internal lumen 408 of the alignment member 402, as shown in phantom in FIG. 7B, whereby the deflectable elongate members 306 of the prosthesis 300 are placed in the compressed configuration;

(iii) after the bone structure prosthesis 300 is loaded into the delivery apparatus 400, the alignment member 402 (with the prosthesis 300 disposed therein) is advanced into the pilot opening in the bone structure; and

(iv) after the alignment member 402 is advanced into the pilot opening, the delivery handle 410 is moved inwardly, i.e., in the direction denoted by arrow “A”, whereby the rod member 412 translates in the same direction within the internal lumen 408 of the alignment member 402, whereby the prosthesis abutment region 414 of the rod member 412 abuts against the bone structure prosthesis 300 and discharges the prosthesis 300 out of the alignment member 402 and into the pilot opening.

According to the invention, when the bone structure prosthesis 300 is discharged out of the alignment member 402 and inserted into the pilot opening, the prosthesis 300 transitions from the compressed configuration shown in FIG. 6A to the expanded configuration, i.e., deflectable elongate members 306 deflect outwardly, as shown in FIG. 6B, and exert forces on the internal surface of the pilot opening, securing the prosthesis 300 in the pilot opening and, thereby, bone structure.

According to the invention, the bone structure prosthesis 300 can comprise various biocompatible materials, including, without limitation stainless-steel, titanium, titanium alloys, cobalt-chromium alloys, tantalum, and magnesium ceramics.

In some embodiments of the invention, the bone structure prosthesis 300 comprises a shape memory alloy.

In some embodiments, the shape memory alloy comprises a superelastic nickel-titanium alloy, e.g., Nitinol (55Ni-45Ti).

In the noted embodiments, the bone structure prosthesis 300 is adapted to undergo a crystal phase transformation from a martensite crystal structure to an austenite crystal structure at a pre-defined transformation temperature and can be deformed (and, hence, shaped) to the expanded configuration shown in FIG. 6B at or above the transformation temperature, stay in the deformed, i.e., expanded, configuration when the force(s) exerted to deform, i.e., shape, the bone structure prosthesis 300 has been removed, transition from the austenite crystal structure back to the martensite crystal structure when the bone structure prosthesis 300 is cooled below the transformation temperature, compressed via an external force or apparatus, such as the deployment apparatus 400, to a compressed configuration; preferably, the compressed configuration shown in FIG. 6A, and then revert (or transition) back to the original expanded configuration upon removal of the external force or released from the apparatus, e.g., deployment apparatus 400.

According to the invention, when the bone structure prosthesis 300 is loaded into the deployment apparatus 400, i.e., the prosthesis 300 is inserted into the internal lumen 408 of the alignment member 402, as shown in phantom in FIG. 7B, and, hence, compressed to a compressed configuration; preferably, the compressed configuration shown in FIG. 6A, and discharged out of the deployment apparatus 400 and inserted into the pilot opening thereafter, the prosthesis 300, i.e., deflectable elongate members 306 thereof, will similarly expand, i.e., the deflectable elongate members 306 deflect outwardly, as shown in FIG. 6B, and exert forces on the internal surface of the pilot opening, securing the prosthesis 300 in the pilot opening and, thereby, bone structure.

According to the invention, the bone structure prosthesis 300 can also comprise various outer coatings.

According to the invention, the outer coating(s) can comprise a composition comprising one of the aforementioned (i) biocompatible polymers, (ii) biocompatible adhesives, (iii) osteogenic agents, and/or (iv) pharmacological agents.

According to the invention, there is thus also provided methods for stabilizing dysfunctional skeletal members, e.g., fractured bones, and dysfunctional bone structures, such as dysfunctional SI joints, comprising (i) providing a suitable bone structure prosthesis of the invention, (ii) creating a pilot opening in the dysfunctional skeletal member or bone structure, and (iii) delivering the bone structure prosthesis to the dysfunctional skeletal member or bone structure.

As will readily be appreciated by one having ordinary skill in the art, the present invention provides numerous advantages compared to prior art apparatus for stabilizing bone structures. Among the advantages are the following:
the provision of improved bone structure prosthesis, which can be readily employed to stabilize dysfunctional bone structures, including individual skeletal members and non-articulating and articulating bone structures; particularly, dysfunctional SI and intervertebral joints;
the provision of improved bone structure prostheses, which, when implanted in a dysfunctional non-articulating or articulating bone structure, such as a dysfunctional SI or intervertebral joint, effectively ameliorate pain associated with bone structure dysfunction;
the provision of improved bone structure prostheses, which, when implanted in a pilot opening in a dysfunctional non-articulating or articulating bone structure, such as a dysfunctional SI or intervertebral joint, exert retaining forces on the internal surface of the pilot opening to secure the prostheses to the pilot opening and, thereby, bone structure;
the provision of improved bone structure prostheses that can readily be employed in minimally-invasive bone structure stabilization methods;
the provision of improved bone structure prostheses that possess optimal structural properties;
the provision of improved bone structure prostheses that can be readily employed to stabilize individual bone structures, i.e., skeletal members, via fixation or fusion; and
the provision of improved bone structure prostheses that facilitate remodeling of damaged osseous tissue and regeneration of new osseous tissue and osseous tissue structures when engaged to bone structures.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

What is claimed is:

1. A bone structure prosthesis comprising an elongated member adapted to be inserted into a pilot opening in a dysfunctional bone structure, said pilot opening comprising an internal surface,
said elongated member comprising a proximal end and a distal end disposed opposite said proximal end, and an expandable mid-region disposed between said proximal and distal ends,
said elongated member further comprising a longitudinal axis,
said expandable mid-region comprising a plurality of deflectable elongate members configured and adapted to transition from a compressed configuration to a deflected configuration, whereby said plurality of deflectable elongate members deflects outwardly in relation to said longitudinal axis of said elongated member, and whereby, when said elongated member is inserted into said pilot opening in said dysfunctional bone structure, said plurality of elongate members exert a retaining force on said internal surface of said pilot opening, whereby said elongated member is secured in said pilot opening and, thereby, said dysfunctional bone structure.

2. The prosthesis of claim 1, wherein said transition of said plurality of deflectable elongate members from said compressed configuration to said deflected configuration is achieved by restraining said elongated member in said compressed configuration in a deployment apparatus and thereafter discharging said elongated member out of said deployment apparatus.

3. The prosthesis of claim 1, wherein said elongated member comprises a shape memory alloy.

4. The prosthesis of claim 3, wherein said shape memory alloy comprises a superelastic nickel-titanium (Ni—Ti) alloy.

5. The prosthesis of claim 4, wherein said transition of said plurality of deflectable elongate members from said compressed configuration to said deflected configuration is induced by said insertion of said elongated member into said pilot opening in said dysfunctional bone structure, whereby said elongated member is subjected to a core temperature of said subject above a crystalline structure transition temperature of said elongated member.

6. The prosthesis of claim 1, wherein said elongated member further comprises an outer coating.

7. The prosthesis of claim 5, wherein said outer coating comprises a biocompatible adhesive composition.

8. The prosthesis of claim 7, wherein said biocompatible adhesive composition comprises a poly(L-glutamic acid)-based composition, poly(γ-glutamic acid)-based composition, poly(alkyl cyano acrylate)-based composition, or polyacrylic acid-based composition.

9. The prosthesis of claim 6, wherein said outer coating comprises an osteogenic composition.

10. The prosthesis of claim 9, wherein said osteogenic composition comprises a poly(glycerol sebacate) (PGS) based composition.

11. The prosthesis of claim 9, wherein said osteogenic composition comprises a bone morphogenic protein (BMP) selected from the group consisting of BMP-1, BMP2a, BMP2b, BMP3, BMP4, BMP5, BMP6, BMP7, and BMP8a.
